(19) United States
(12) Patent Application Publication
(10) Pub. No.: US 2020/0129115 A1
(43) Pub. Date: Apr. 30, 2020

(54) IMPROVED DIAGNOSTIC INSTRUMENT AND METHODS

(71) Applicants: BROWN UNIVERSITY, Providence, RI (US); WOMEN & INFANTS HOSPITAL OF RHODE ISLAND, Providence, RI (US)

(72) Inventors: Barry M. LESTER, Warwick, RI (US); Stephen J. SHEINKOPF, Barrington, RI (US); Harvey F. SILVERMAN, East Greenwich, RI (US)

(21) Appl. No.: 16/500,921

(22) PCT Filed: Apr. 6, 2018
(86) PCT No.: PCT/US18/26419

§ 371 Date: Oct. 4, 2019

Related U.S. Application Data
(60) Provisional application No. 62/482,483, filed on Apr. 6, 2017.

(51) Int. Cl. A61B 5/00; G10L 25/18; G10L 25/63; G16H 50/30; G16H 50/20

(52) U.S. Cl. CPC A61B 5/4803; G10L 25/18; G10L 25/63; A61B 2503/045; G16H 50/20; A61B 5/7267; G16H 50/30

(57) ABSTRACT

An Infant Cry Analyser (ICA) records cries of a neonate and applies acoustic analysis to identify features characteristic of neonatal abstinence syndrome (NAS) or drug withdrawal. The ICA may also receive non-acoustical input, data, such as a medication history, sleep history or other potentially relevant data, and may be configured to output a medical record of the detected data, diagnostic acoustic characteristics or ultimate diagnosis, in one embodiment, the ICA is used on populations of infants to develop normative measures of the characteristics of the infant cries, of the target population, e.g., is used on identified sub-populations such as neonates having laboratory findings of drugs in body fluids, to develop NAS diagnostic criteria or tables. The ICA produces repeatable acoustic measures that reflect, and allows early identification of, other conditions or pathologies in which a cry or vocalization is distinctive of an underlying condition or pathology.

### RELATED APPLICATION

This application claims priority to U.S. provisional application Ser. No. 62/482,483, filed Apr. 6, 2017, the entire disclosure of which is incorporated herein by reference.

### BACKGROUND

The world is currently experiencing an opioid epidemic, characterized by widespread use of opioids and their pharmacological derivatives and analogs, as well as addictive behaviors and physical traits developed upon exposure to these compounds and preparations. Neonatal Abstinence Syndrome (NAS) (also known as substance withdrawal disorder) is a withdrawal that occurs in newborn infants who have experienced prenatal exposure and is characterized by a constellation of behaviors and conditions. NAS behaviors may surface upon birth and the abrupt discontinuation of prenatal exposure to opiates, including substances such as prescription pain medication given to the mother. The symptoms may require administration of opiate treatment drugs, and phased withdrawal from the treatment over the first few weeks of life.

Current methods to diagnose NAS rely heavily on cry characteristics (e.g., pitch, duration, quality of crying and irregular vocalizations) as well as a number of physiological and behavioral traits including feeding and sleeping patterns, muscle spasms, metabolic, vasomotor, respiratory or gastrointestinal disturbances, unusual vocalization stresses and other symptoms. When a history of in utero drug exposure is suspected, neonatal protocols may call for observation to confirm the condition, and assessment of multiple traits every two or four hours during the days immediately following birth.

However, the usual descriptions of these traits are neither well-defined, nor commonly understood, and are not clearly communicable descriptions and rules for recognition or management by clinic staff. Moreover, determination of the diagnostic indicators by hospital staff can be highly subjective and could lead to the misdiagnosis of, or failure to diagnose NAS possibly resulting in poor or inappropriate treatment.

The opioid epidemic has called for more objective measures of NAS that is a significant public health problem and healthcare burden that has increased by over 380% in the United States between 2000-2012 due, in part, to the use and abuse of prescription opioids for pain management during pregnancy. The total costs of NAS have been estimated to exceed $1.5 billion per year. The bulk of these costs are hospital related due to prolonged hospitalization, typically 15 days to over three months, and are borne by state Medicaid programs. NAS is a highly prevalent condition that has cast a spotlight on the need for an accurate diagnosis.

Accurate diagnosis is critical because a positive diagnosis triggers pharmacological treatment in which an opioid (e.g. morphine) is reintroduced and the infant is weaned until no longer symptomatic. The prolonged hospitalization results from the length of time of the weaning process. Misdiagnosis can lead to mismanagement of infants who should or should not be treated. Unfortunately, NAS has thus far defied conventional approaches to management because current protocols and guidelines are not evidence-based. Yet, tas the prevalence of opioid use during pregnancy continues to increase, NAS will increase to reflect use/abuse in the society at large. The development of an objective diagnostic test and monitoring instrument would therefore be an improvement.

### SUMMARY

The invention provides an automated, computerized Infant Cry Analyzer (ICA) that quantifies infant crying by measuring objectively defined acoustic characteristics of infant cries. The acoustic cry analyzer provides a reliable, objective measure of some acoustical properties of the cry, allowing the acoustic frequencies, duration and composition of sound bursts, power of individual vocalizations, as well as the power of each cry or cry interval to be objectively measured and displayed. This collection of parameters that are directly detected and displayed for each recorded cry are intended to detect and present objective measures or characteristic spectral representations for a number of infant cry traits that have previously been defined by rather informal clinical descriptors. The ICA can be configured to monitor and record cries, compile a database and measure or analyse the properties of a cry relevant for diagnostic or patient monitoring purposes. For example, by collecting data records for a control group of neonates—for example, ones who have exhibited detectable levels of opioid metabolites in body fluid—and applying artificial intelligence to characterize the datasets of NAS infants, the instrument can provide an NAS spectrum for simple, early and accurate diagnosis and clinical management of NAS infants.

Initially, several measurements are taken, and the acoustic cry analyzer is used to quantify the acoustic characteristics of cries from different infant populations and to identify or refine the diagnostic acoustical spectra for NAS infants. Suitable populations for the development protocol include a group of infants diagnosed with NAS and a group of healthy infants, to which they are compared. Embodiments of the invention distinguish NAS acoustic characteristics from normal cry data. Further embodiments compile correlations with other clinical observations such as tremors, spasms, body temperature, post-feeding quiet interval and other indicators. These may include characteristics such as those listed in the widely-accepted Finnegan NAS diagnostic chart (Finnegan LP. Neonatal abstinence syndrome: assessment and pharmacotherapy. In: Nelson N, editor. Current therapy in neonatal-perinatal medicine. 2 ed. Ontario: BC Decker; 1990.), to develop a fast, objective and more effective diagnosis. The acoustic cry analyser can be incorporated into a smartphone, tablet computer, personal computer, or an automated, hand-held “iPhone®-like” device programmed to record sound, process the recording to identify objectively relevant and measurable characteristics of the acoustic spectrum, and provide a digital diagnostic readout indicative of whether or not the infant's cry is symptomatic of NAS. In one embodiment, the instrument can also allow user entry of certain non-acoustic screening data (such as medication history and lab analysis of body fluids), to produce a definitive diagnoses, and/or may print a spreadsheet-style medical record which includes the entered data, detected cry data and NAS Finnegan score.

Operation of the instrument is refined using field data to establish baseline characteristic properties of infant cries, allowing faster identification of the acoustic signature for NAS diagnosis. According to this aspect of the invention, an initial stage involves collecting normal, suspected NAS and confirmed NAS infant cry data, and incorporating the corresponding measures, thresholds or acoustic features in one or more reference tables. These are then used to automatically analyze infant sounds and to provide a more accurate diagnosis of NAS. Such representative sound recognition tables are also used to acoustically monitor the stages of withdrawal following birth and until the infant is ready for release. This reduces the likelihood of misdiagnosis, and promotes early recognition, and better assures adequate treatment and efficient management of these infants.

Further description of the diagnostic device of the invention, procedures for determining a number of relevant measurable acoustic traits of an infant cry, as well as illustrative records, such as spectra and energy diagrams for automated and analytically-derived measures of relevance employed in the instrumented diagnostic procedure appear infra. For a general understanding of the instrument and acoustic technologies, reference is made to a cry acoustics analysis instrument previously developed for early detection of autism or Asperger syndrome, and the acoustic characteristics of infant cries involved in that inquiry, as described in earlier-filed international patent application serial number PCT/US2013/057295, filed Aug. 29, 2013 and published as US 2015/0265206, and U.S. provisional application Ser. No. 61/694,437, filed Aug. 29, 2012, and Ser. No. 61/718,384 filed Oct. 25, 2012 entitled, “Accurate analysis tool and method for the quantitative acoustic assessment of infant cry” by inventors Stephen J. Sheinkopf, Barry M. Lester and Harvey F. Silverman, each of which is hereby incorporated herein by reference in its entirety, provide background understanding of certain acoustical analysis methodologies that can be applied for producing device-recognizable acoustic data objects of the present invention, and illustrating spectral analysis or processing procedures for cry-derived measurements and production of clinical conclusions which in those references are directed to early detection of Autism Spectrum Disorder (ASD). A number of published technical papers are listed in the aforemtioned patent applications, and those papers are also incorporated herein by reference in their entireties. In addition, certain Figures described further below graphically illustrate: stages of cry collection; show several spectral parameters of relevance; and describe development of an automated, hand-held clinical recorder/diagnose instrument with appropriate threshold and other processing schemata to quickly detect, determine or refine the definitions of diagnostically-relevant cry traits for the NAS condition of the present invention.

Thus, the invention provides for the use of the infant cry analyzer (ICA) for detection of a cry “signature” in neonates and infants indicative of neonatal abstinence syndrome (NAS or opiate withdrawal syndrome) to improve accuracy and detection of NAS.

### BRIEF DESCRIPTION OF THE DRAWINGS

The invention will be understood from the description and claims below, taken together with the drawings wherein:

FIG. 1 shows infant cry spectrographs for two cries recorded over the course of post-delivery and discharge interval;

FIG. 2 shows mean fundamental frequency and scores on the Finnegan NAS diagnostic chart for eight cry samples;

FIG. 3 shows mean amount of frication and Finnegan scores for the eight cry samples; and

FIG. 4 illustrates a machine-learning process by which ICA measurements on identified groups of normal and of NAS infants determine acoustic spectra for automated diagnosis and ongoing evaluation of NAS infants.

### DETAILED DESCRIPTION

The current “gold standard” used to diagnose NAS is the Finnegan scale, a multi-component assessment that produces a numerical score based upon the number of NAS-related symptoms exhibited by the infant. Symptoms include central nervous system hyperirritability, and dysfunction of the autonomic nervous system, gastrointestinal tract, and respiratory system based on medical chart review (e.g., amount of sleep), and direct observation (e.g., tremors), typically completed by nurses. The diagnosis of NAS is made when the Finnegan score reaches a predefined numerical threshold.

A number of concerns have been raised about the Finnegan scale including the subjective nature and inadequate definition of some of the items (especially crying, the focus of this description), amount of time to administer, and poor agreement on scoring the items among the nurses who administer the scale. Perhaps most alarming is research showing that the Finnegan scale has poor psychometric properties that jeopardize its reliability and validity and raise questions about its use for the diagnosis and treatment of NAS. In fact, dissatisfaction with the Finnegan scale has led to attempts to modify the scale to the point where local variants of the measure are used more frequently than the original scale itself.

Crying is prominent in the diagnosis of NAS on the Finnegan scale and, unfortunately, is one of the most poorly measured symptoms.⁵ Crying related symptoms of NAS on the Finnegan scale include 1) excessive high pitched cry, 2) high pitched at its peak, 3) high pitched throughout or 4) prolonged crying even if not high pitched. This is a highly subjective definition based on nurses' perceptions of acoustic measures rather than direct quantification of acoustic measures. Most critically, a high-pitched cry is scored “when the infant is unable to decrease crying within a 15 second period . . . or if the infant continues to cry intently or continuously for up to 5 minutes . . . if these signs are present this item (excessive high-pitched cry) should be scored whether the infant's cry is high pitched or not”.

Embodiments of the present invention improve the psychometric properties of the Finnegan scale by providing a specialized acoustic recorder/sound analyser. Other embodiments of the present invention provide a hand-held or portable or automated recorder or analyzer programmed to detects relevant acoustic features in recordings of an infant to improve the diagnosis of NAS.

The infant cry analyzer (ICA) of the present invention enables precise measurement of these and other potential acoustic properties of cries in infants with NAS, so as to define, or develop, or refine an NAS “cry signature”. For example, crying in infants with NAS has also been described as a “pain” cry, which could both be part of the NAS cry signature and also have unique acoustical characteristics for use in other venues. For example, the ability to quantify the acoustic characteristics of a pain cry could lead to the development of a companion device for pain detection in infants, an area that currently also lacks an objective basis.

There is mounting concern that the lack of objective measures of NAS could lead to the misdiagnosis of NAS; both false positive and false negative findings would result in inadequate or inappropriate treatment and affect infant outcome, and significantly affect the required length of stay (LOS). The ICA addresses this issue by improving the measurement of the critical crying components and psychometric properties of the Finnegan scale, allowing the preparation of objective diagnostic criteria which can potentially reduce LOS in infants with NAS.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like), non-volatile memory (e.g., read-only memory, hard disks, floppy disks, magnetic tape, optical discs, paper tape, punch cards, and the like). The computer processor can be a component of a device such as a smartphone (e.g., a device sold under the IPHONE® trademark by Apple, Inc. of Cupertino, Calif., the WINDOWS® trademark by Microsoft Corporation of Redmond Wash., the ANDROID® trademark by Google Inc. of Mountain View, Calif., and the like), a tablet (e.g., devices sold under the IPAD® trademark from Apple Inc. of Cupertino, Calif. and the KINDLE® trademark from Amazon Technologies, LLC of Reno, Nev. and devices that utilize WINDOWS® operating systems available from Microsoft Corporation of Redmond, Wash. or ANDROID® operating systems available from Google Inc. of Mountain View, Calif.), a personal computer (e.g., a laptop of a desktop computer), a server, and the like.

Additionally or alternatively, the methods described herein can be implemented in computer hardware such as an application-specific integrated circuit (ASIC).

### Example

An ICA in accordance with the invention was constructed using “state of the art” cepstral analysis to extract acoustic parameters from cry recordings outputted to standard audio files. One related cry analysis instrument was described in an earlier international patent application, published as WO2014/036263 entitled A Flexible Analysis Tool for the Quantitative Acoustic Assessment of Infant Cry. Reference is made to that document, the entire disclosure of which is incorporated herein by reference, for certain principles of construction and operation. That instrument was set up to identify cries symptomatic of autism vocalizations, and its accuracy for recognizing acoustic features was evaluated and compared to manual coding of pitch periods (fundamental frequency or F0) and voiced segments of cries from spectrographic displays. In the present development, the ICA is used to objectively analyze the acoustics of infant populations for traits associated with NAS to better define the characteristics of NAS and normal infants, and to distinguish between the two conditions.

An infant's cry is a sequence of utterances and silences. An utterance is a contained vocal output, either voiced (generated via vocal vibrations for which the pitch or frequency is detected) or unvoiced (due to frication or tension in the vocal tract). The ICA accepts digitized infant cry recordings as input, which is then classified as utterances, or silence (amount of time between utterances). Acoustic parameters are calculated for each sound segment. The ICA is unique from other speech analyzers because it applies current digital signal processing techniques and is specifically tailored to infant acoustic data, i.e. acoustic parameters that are sensitive to the developing vocal tract and oral cavity of an infant.

In accordance with the ICA and methods described herein, the cries of a newborn with prenatal opioid exposure were recorded at a local hospital. These cries were recorded during the administration of the Finnegan scale at 8 time points until the infant was discharged from the hospital several weeks after birth. The infant was diagnosed with NAS based on the Finnegan scale and treated with pharmacological intervention for 23 days. The ICA acoustic analysis of these cries was compared with the contemporaneous nurse scoring of the cry components on the Finnegan scale (Table 1).

TABLE 1
Cry Features From Case Study of Infant Treated for NAS During Hospital Stay

| Cry Feature | Cry 1 | Cry 2 | Cry 3 | Cry 4 | Cry 5 | Cry 6 | Cry 7 | Cry 8 |
|---|---|---|---|---|---|---|---|---|
| Average Pitch | 486.9 | 452.5 | 473.5 | 496.1 | 553.1 | 487.4 | 623.5 | 438.5 |
| (F0 in Hz) | | | | | | | | |
| Maximum Pitch | 680.4 | 592.0 | 655.0 | 700.6 | 709.6 | 636.7 | 869.8 | 621.3 |
| (F0 in Hz) | | | | | | | | |
| Frication (proportion) | 0.72 | 0.64 | 0.47 | 0.51 | 0.63 | 0.55 | 0.38 | 0.2 |
| Average Energy | 68.5 | 66.6 | 66.4 | 68.5 | 70.0 | 82.1 | 78.8 | 70.9 |
| (dB) | | | | | | | | |
| Maximum Energy | 73.0 | 72.2 | 71.3 | 73.0 | 73.9 | 84.4 | 83.5 | 74.2 |
| (dB) | | | | | | | | |

On the Finnegan scale, the infant was scored as having an Excessive High Pitched Cry during the first (Cry 1) and third (Cry 3) days on which the cry was also recorded. The average pitch of the cries on days 1 and 3 was not substantially different from the cries on any of the other days (Table 1). Moreover, none of these cries met the usual definitions of high pitch, which is typically considered to be above 800 Hz or above 1000 Hz. On the Finnegan rating, NAS symptoms start when the total score reaches 8 or above. The Finnegan score was <8 for Cry 1 and was 8 for Cry 3. If Excessive High Pitched had not been scored for Cry 3, the Finnegan score would have been <8. This may indicate a potential misdiagnosis, in judging the infant to be symptomatic when he was not. In order to bring objectivity to the determination of NAS, the ICA was used to objectively analyze the cry spectra.

The scoring anomaly raises the question of what nurses are actually perceiving when they rate a cry as high-pitched. One possibility is that they are reacting to the maximum pitch (which could be a momentary spike that is higher than the average pitch) shown Table 1. Frication and energy (Table 1) are other characteristics that have been implicated. Another possibility is that these cries were not perceived as high pitched but were scored as high pitched simply based on the Finnegan scoring instructions/criteria for crying mentioned above in which excessive high-pitched cry is scored whether the cry is high pitched or not if the infant has other cry related problems (such as inconsolability).

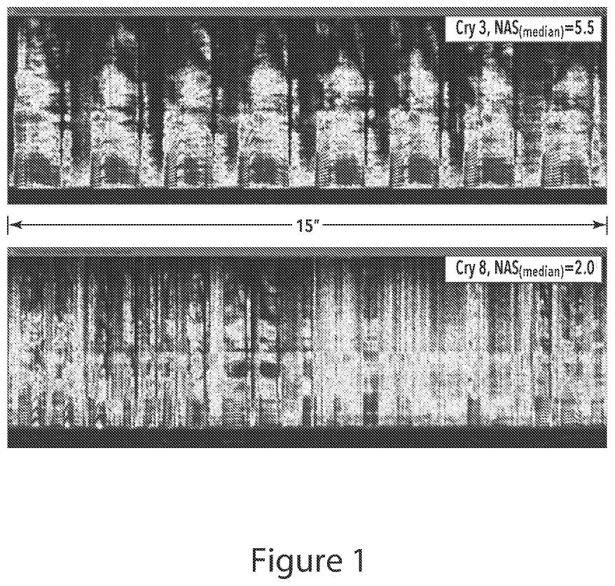

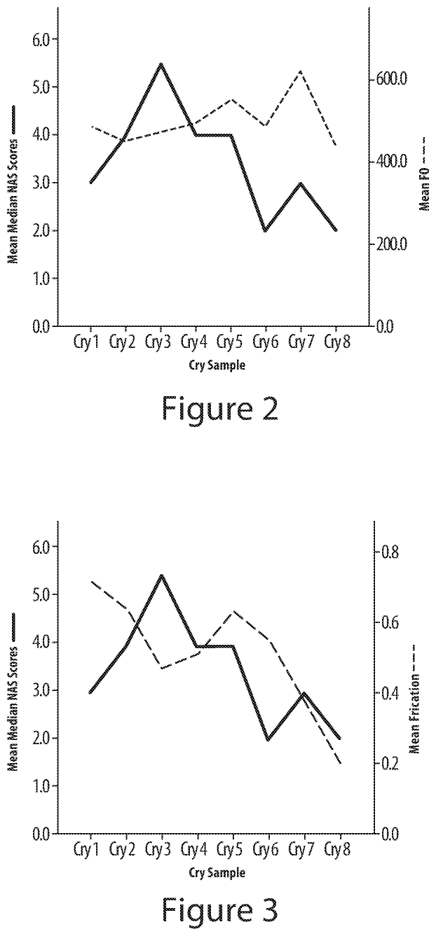

FIG. 1 illustrates spectrographs of cry sample 3 taken on day 11 when the NAS symptom rating was high, and cry sample 8 on day 24 when the NAS symptoms were resolving. More generally, FIG. 2 and FIG. 3 show the fundamental frequency F0 and amount of frication, respectively, in each of the cry samples 1-8, plotted against the NAS score, to better visualize the relationship between perception of acoustic cry properties and NAS scoring.

There is also a possible role of pain cry characteristics as part of the NAS cry signature. Moreover, the identification of a pain cry alone could have clinical, scientific and commercial applications beyond NAS for other aspects of diagnosing infant health or injury, and the initial calibration and interpretation of ICA-recorded cry spectra is expected to lead to further diagnostic utility for management of neonates. However, in the instant example, it is noteworthy that the Cries 3 and 8 above (shown in FIG. 1) “look” respectively like acoustic spectra of pain and non-pain cries as evidenced by the longer utterance lengths and longer intervals between utterances in Cry 3 vs. Cry 8, so that the naïve listeners described Cry 3 as “abnormal” and Cry 8 as “healthy” based upon their perception of these pain vs. non-pain characteristics in reaching that description. This phenomenon could also underlie the cry ratings reported by a nurse applying the Finnegan scale.

Notably, as shown in FIGS. 2 and 3, the fundamental frequency actually rises as withdrawal symptoms recede, although the NAS score rises, and frication is highest initially and at one or more intermediate times. Further observations were deemed necessary to determine a definite NAS cry signature based on the ICA data sets. This is done with observations using the ICA to determine acoustic cry characteristics that differ between infants with NAS and infants without NAS (controls). The major cry characteristics of interest are the fundamental frequency (the base frequency of a cry that is perceived as pitch), frication (tension in the vocal tract that can be described as strident), dysphonation (unvoiced periods of cry perceived as “noise” or distortion), decibel level (loudness), and timing measures (amount of time (seconds) of each vocal component and amount of time between each vocal component). Additional acoustic measures will also be explored.

A machine learning approach is then applied using these acoustic characteristics to recognize an NAS cry signature that can classify individual infants into those with a unique cry symptomatic of NAS versus those whose cry is not symptomatic of NAS.

Methods.

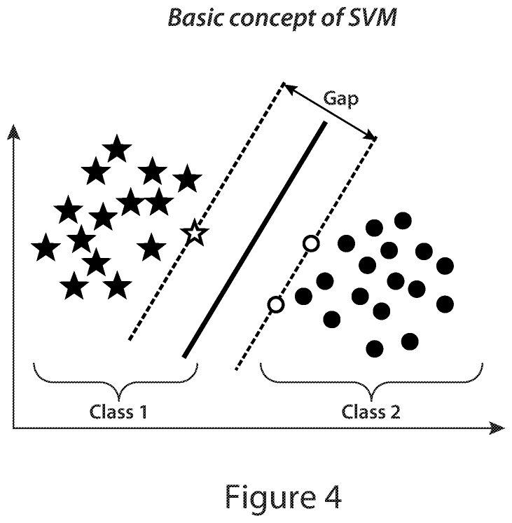

Crying will be recorded at the hospital from 3 groups of infants, infants diagnosed with NAS (n=45) based on the Finnegan scale, drug-exposed infants not diagnosed with NAS (n=23), and normal, healthy infants (n=50) with no prenatal exposure to opioids or other substances of abuse (e.g., cocaine, methamphetamine, marijuana, tobacco or alcohol). The sample size of the NAS group is based on the incidence of NAS at the hospital. Currently, approximately 8 infants per month are born with prenatal opioid exposure, or 80 infants during our intended enrollment period. Of these 80 infants, 85% (n=68) consent to participate because no additional procedures are added in as much as the cry of the participating infant is only recorded when the Finnegan is administered, which is part of hospital standard care. Based on current statistics, ⅔ (n=45) of the infants will develop NAS and n=23 will be opioid exposed but not develop NAS. The collected acoustic data is analyzed by a supported vector machine learning approach. This is illustrated schematically in FIG. 4.

The infant's cry is recorded during the administration of the Finnegan scale when the Finnegan scores reach a diagnostic threshold but before treatment is initiated, enabling analysis of the “NAS” cry. For the control group, crying is recorded before hospital discharge during routine handling such as diaper changes, bathing or just before feeding. The Cepstral based ICA is used to extract acoustic parameters from the cry recordings. Differences in acoustic cry characteristics between infants with NAS and non-exposed infants are examined using generalized estimating equation (GEE) models. Using GEE takes into account clusters of observations and accounts for variation in correlation from the use of repeated outcome measures. The efficiency is then determined of a computer-based algorithm to recognize an NAS cry signature that can classify individual infants into those with a unique cry symptomatic of NAS versus those whose cry is not symptomatic of NAS. Such a classifier is expected to rely on patterns amongst the range of acoustic features that differ between the NAS and control groups. The decision-making algorithm is based on the Supported Vector Machine (SVM) machine-learning approach that iteratively refines algorithms using training and validation data sets from the infants in the NAS and control groups.

The basic concept of the SVM approach is shown in FIG. 4. Given a set of training examples, each marked as belonging to one or the other of two categories, an SVM training algorithm builds a model that assigns new examples to one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall. Receiver operating characteristic curves based on the accuracy of our algorithm to correctly classify infants as NAS or controls will be constructed. Optimal cutoff values will then be determined using the maximum Proportion Correctly Classified. Sensitivities, specificities, and positive and negative predictive values will also be calculated.

Power Analysis.

For acoustic cry characteristics, given an alpha level of 0.05, with 45 NAS infants, 23 opioid exposed without NAS and 50 non-exposed infants, a power of 86% detects medium to small effect sizes in GEE models.

Another objective is to determine improvement in the psychometric properties of the Finnegan scale with the inclusion of the ICA cry signature. The Finnegan scale has poor psychometric properties that jeopardize its reliability, validity and use for the diagnosis and treatment of NAS. We hypothesize that psychometric properties of the Finnegan scale (internal consistency and item correlations) will be significantly higher using the ICA cry signature compared with the Finnegan cry measures (excessive high pitched cry, high pitched at its peak, high pitched throughout or prolonged and inconsolable even if not high pitched). This hypothesis is supported above in Preliminary Study from the infant with NAS suggesting discrepancies between the nurses' cry ratings on the Finnegan and our acoustic measures of the same cry.

For this determination, the individual items and total Finnegan scores and ICA cry signature data collected at the same point in time are used as described in Objective 1 above, when the Finnegan scores reach a diagnostic threshold but before treatment is initiated. Two Finnegan scale scores are computed, the Finnegan score using the current Finnegan cry measures and using the Finnegan-ICA in which the Finnegan cry measures are replaced with the ICA cry signature measure. Item total correlations are calculated for the Finnegan Scale and the Finnegan-ICA scale. Cronbach alpha, a measure of internal consistency which represents how closely a related set of items are as a group, is also calculated for each scale and compared using the Feldt test.

Power Analysis.

For the correlation of the Finnegan items, given an alpha level of 0.05, with 42 NAS infants, 22 exposed infants, and 50 non-exposed infants, a power of 0.93 is used to detect medium to small effect sizes.

Improvement in infant outcome when the ICA cry signature is included in the diagnosis of NAS is determined. Differences in length-of-stay (LOS) when scores on the Finnegan indicate NAS vs when scores on the Finnegan-ICA indicate NAS are also determined. Although not wishing to be bound by theory, infants with a positive diagnosis for NAS on the Finnegan scale and a negative diagnosis on the Finnegan-ICA will have the longest LOS. This suggests that infants are more likely to be misdiagnosed with NAS using the Finnegan scale and that the use of the Finnegan-ICA can reduce the number of infants who are misdiagnosed.

Methods.

LOS is calculated for all infants. Mean LOS is compared between infants with Finnegan scores >8 and Finnegan-ICA scores <8 using one-way ANOVA. Odds Ratios are used to determine the likelihood of a longer LOS in infants with Finnegan scores >8 and Finnegan-ICA scores <8.

Power.

For logistic regression models, given a conservative alpha level of 0.01 and adjusting for the potential influence of covariates on 45 NAS infants, 23 exposed infants, and 50 non-exposed infants, a power of 0.90 is to detect medium effects.

Another aspect of the invention provides an automated, hand held “iPhone-like” device that will provide a digital readout indicative of whether or not an infant's cry is symptomatic of NAS. This information can then be used to provide a more accurate diagnosis of NAS, thereby reducing the likelihood of misdiagnosis, and improve the treatment and management of these infants.

Another aspect of the invention provides a computer-implemented method for diagnosing Neonatal Abstinence Syndrome in an neonate or infant. The method includes the steps of filtering a digital recording of an infant cry to produce a first filtered digital signal, estimating a fundamental frequency and a cepstrum value of the infant cry by applying to the first filtered digital signal an inverse discrete Fourier transform to obtain the fundamental frequency and cepstrum estimate value of the first filtered digital signal, thereby obtaining a second filtered digital signal, and applying a previously trained classification algorithm to the second filtered digital signal. In one embodiment, the previously trained classification algorithm is a support vector machine.

The invention being thus disclosed and described, further properties and advantageous methods of use and variations will occur to those skilled in the art and understood from the description herein and claims appended hereto.

1. A method for detecting and monitoring Neonatal Abstinence Syndrome (NAS) in a neonate or infant, comprising performing an automated analysis of spectral traits of a cry or crying behavior of identified NAS infants and normal infants to develop an automated measure for acoustic diagnosis of NAS, thereby detecting and monitoring NAS in a neonate or infant.

2. The method of claim 1, further comprising:
identifying a set of spectral characteristics of acoustical measurements of an infant cry recording by inspecting spectral traits of cry recordings of groups of normal infants, of infants diagnosed with NAS and of infants at-risk for NAS;
determining the objective characteristics of demonstrable diagnostic value; and
determining features of the acoustic spectrum of an infant cry that are objectively correlated with either presence or absence of NAS.

3. The method of claim 1, wherein the detection or monitoring is performed by a recording and/or analysis instrument programmed to detect positive diagnosis and/or degree of NAS in a neonate or infant.

4. The method of claim 1, wherein the automated analysis is performed by a hand-held or small device that records cries and analyzes spectral components forming a distinctive NAS signature, and the device is programmed to monitor stage of withdrawal and determine when it is safe or appropriate to discharge the neonate or infant from the hospital.

5. The method of claim 1, wherein the spectral traits include duration, frication and fundamental frequency in the spectral traits in an infant cry which are indicia of strain or pained vocalization, or other spectral features corresponding to recognizable auditory cry descriptors, wherein said the spectral traits have objective analytic basis and are subject to automated analysis to provide a diagnosis not limited by experience or inexperience of a care-giver.

6. A device for clinical diagnosis or monitoring, comprising:
an acoustic monitor programmed to detect and record neonatal cries, and
an acoustic analysis application operative on the cries or recorded cries to determine cry interval, duration, and diagnostic spectral traits effective to detect NAS or monitor intensity or change of an NAS condition.

7. A device for diagnosis of neonatal abstinence syndrome comprising:
an acoustic monitor programmed to detect and record neonatal cries, or process recorded cries, and
an acoustic analysis application operative to process recorded cries to determine cry interval, cry duration, frequency, frication or other spectral characteristics effective to detect NAS or monitor intensity or change of withdrawal symptoms.

8. A computer-implemented method for diagnosing Neonatal Abstinence Syndrome in an neonate or infant, the computer-implemented method comprising:
filtering a digital recording of an infant cry to produce a first filtered digital signal;
estimating a fundamental frequency and a cepstrum value of the infant cry by applying to the first filtered digital signal an inverse discrete Fourier transform to obtain the fundamental frequency and cepstrum estimate value of the first filtered digital signal, thereby obtaining a second filtered digital signal;
applying a previously trained classification algorithm to the second filtered digital signal.

9. The computer-implemented method of claim 7, wherein the previously trained classification algorithm is a support vector machine.
